(19) United States
(12) Patent Application Publication
(10) Pub. No.: US 2019/0302078 A1
(43) Pub. Date: Oct. 3, 2019

(54) Sensor for the Direct Detection of Iodine

(71) Applicant: National Technology & Engineering Solutions of Sandia, LLC, Albuquerque, NM (US)

(72) Inventors: Leo J. Small, Albuquerque, NM (US); Tina M. Nenoff, Albuquerque, NM (US)

(21) Appl. No.: 15/937,298

(22) Filed: Mar. 27, 2018

(51) Int. Cl. G01N 33/00; G01N 27/12; G01N 27/04

(52) U.S. Cl. CPC G01N 33/0055; G01N 27/041; G01N 27/125; G01N 33/0031

(57) ABSTRACT

An all solid-state, MOF-, zeolite-, or activated carbon-based electrical readout sensor with a long-lived signal can be tuned specifically for real-time sensing of iodine gas in ambient conditions. The sensor may be of use in nuclear accident scenarios for first responders and/or as process sensors in advanced nuclear fuel recycling.

### STATEMENT OF GOVERNMENT INTEREST

This invention was made with Government support under Contract No. DE-NA0003525 awarded by the United States Department of Energy/National Nuclear Security Administration. The Government has certain rights in the invention.

### FIELD OF THE INVENTION

The present invention relates to chemical sensors and, in particular, to a sensor for the direct detection of iodine.

### BACKGROUND OF THE INVENTION

Nuclear energy is considered a plentiful source of greenhouse gas free energy. However, safety associated with it is paramount. In the case of accidents or advanced nuclear fuel recycle processes, capture and detection of radiological off-gases is required. See B. J. Riley et al., J. Nucl. Mater. 470, 307 (2016). One main gas of concern is radiological iodine gas (I₂), whose two isotopes of interest include ¹²⁹I, with a ˜17 million-year half-life, and ¹³¹I, with a short 8-day half-life, but which has a strong negative activity in human metabolic processes. In the case of accidents, a quick fail-proof detection response is needed for the safety of first responders and notification of the nearby population. Combining strong electrical readout responses with highly iodine selective materials (in the presence of both air and fission gases) in a detection sensor would greatly protect people in the area.

Metal-organic frameworks (MOFs) have tunable nano- to mesopores that have been an area of intensive research for their highly selective and high capacity gas sorption properties. See J.-R. Li et al., Chem. Soc. Rev. 38, 1477 (2009); and S. Ma and H.-C. Zhou, Chem. Commun. 46, 44 (2010). Various nanoporous materials, such as zeolites and MOFs, have been studied for the bulk sorption of iodine species (such as I₂ and CH₃I) from a variety of gas streams. See K. W. Chapman et al., J. Amer. Chem. Soc. 132, 8897 (2010); D. F. Sava et al., J. Amer. Chem. Soc. 133, 12398 (2011); T. M. Nenoff et al., Micro. Meso. Mater. 200, 297 (2014); J. T. Hughes et al., J. Amer. Chem. Soc. 135, 16256 (2013); D. F. Sava Gallis et al., Ind. Eng. Chem. Res. 56, 2331 (2017); and D. F. Sava et al., Chem. Mater. 25, 2591 (2013), which are incorporated herein by reference.

Specific to sensing devices, MOFs have been widely used to successfully sense a range of gases via various optical techniques. See L. Kreno et al., Chem. Rev. 112, 1105 (2012); G. Lu and J. Hupp, J. Amer. Chem. Soc. 132, 7832 (2010); D. Ma et al., Chem. Commun. 49, 8964 (2013); S. Sanda et al., Chem. Commun. 51, 6576 (2015); and Y. Xiao et al., Chem. Commun. 46, 5503 (2010). However, there are drawbacks with optical readouts, including added machinery and associated complexity to deliver the signal from the field. By contrast, direct electrical devices are preferred in the field because of their simplicity of measurement readout, lack of costly expenses, and high reliability that is integrated into modern electronics. See V. Stavila et al., Chem. Soc. Rev. 43, 5994 (2014).

Gas sensing by electrical readout devices do exist. However, their fundamental nature has drawbacks for ambient air-based fission gas sensing. Currently, electrical conductivity-based devices generally fall into two categories. They include solid-state oxide-based devices and fuel cell devices. The former requires higher temperatures (>200° C.) for interaction of the gas with the surface oxides and thus require heating devices attached to the sensor. The latter are room temperature devices with liquid electrolytes, which suffer from fouling and short lifetimes.

Impedance spectroscopy (IS) is a valuable tool for determining the electrical response of multiphase materials systems. It has been successfully used to characterize a range of systems, including batteries, the assembly of monolayers on surfaces, the molecular orientation of liquids at interfaces, and charge transfer process in MOFs. See N. Hudak et al., J. Electrochem. Soc. 162, A2188 (2015); I. Escalante-Garcia et al., J. Electrochem. Soc. 162, A363 (2015); C. Saby et al., Langmuir 13, 6805 (1997); L. J. Small et al., Langmuir 30, 14212 (2014); L. J. Small and D. R. Wheeler, J. Electrochem. Soc. 161, H260 (2014); T. Pajkossy and R. Jurczakowski, Curr. Opin. Electrochem. 1, 53 (2017); E. Spoerke et al., J. Phys. Chem. C 121, 4816 (2017); and D. Lee et al., J. Phys. Chem. C 118, 16328 (2014). Fundamentally, IS applies a small sinusoidal voltage to a sample and measures the resulting complex current, with “impedance” being defined as the ratio of the complex voltage to the complex current. See E. Barsoukov and J. Macdonald, Impedance Spectroscopy: Theory, Experiment, and Applications, 2nd ed.; Wiley, 2005. As the frequency of the sine wave is varied, processes unique to a given frequency range can be observed. This allows for the isolation and quantification of specific processes from the overall system response. Impedance spectroscopy is particularly advantageous in its ability to measure processes in real time without influencing the system, as the AC voltage is small and the net current can be made zero. With proper instrumentation, very large impedances (e.g., 10¹⁴Ω) can be readily and accurately measured.

The attractiveness of IS has not gone unnoticed in the sensor community. Several groups have used a variety of MOFs and zeolites to construct IS-based sensors for detection of various hydrocarbons, water, or alcohols. Several of these sensors operated at elevated (120-350° C.) temperatures. See S. Reib et al., Sensors 8, 7904 (2008); S. Achmann et al., Sensors 9, 1574 (2009); K. Alberti and F. Fetting, Sensor Actuat. B 21, 39 (1994); G. Hagen et al., Sensor Actuat. B 119, 441 (2006); and P. Kurzweil et al., Sensor Actuat. B 24-25, 653 (1995). These sensors display good selectivity to the target gas. However, nearly all measured changes in impedance were within the same order of magnitude. This constrained the limit of detection, thereby requiring precision equipment to detect the subtle changes in impedance (e.g., sub-pF or 10¹⁰Ω range).

Recognizing the limitations imposed by many MOFs' high impedances, several groups have taken advantage of their porosity and incorporated designer organic molecules or I₂ to tune their electrical conductivities. See D. Lee et al., J. Phys. Chem. C 118, 16328 (2014); A. Talin et al., Science 343, 66 (2014); G.-P. Li et al., ChemPlusChem 82, 716 (2017); and M.-H. Zeng et al., J. Amer. Chem. Soc. 132, 2561 (2010). In the latter reports, I₂ doping was constrained to the liquid phase. This is not optimal for many real-world applications. Long-lived signals in durable sensors require an all solid-state construction capable of detecting gaseous I₂. By contrast, there has been recent sensor development in which redox active MOFs had their conductivity temporarily altered by adsorbed I₂ gas. However, the response readily reverted back when exposed to air. See Y. Kobayashi et al., Chem. Mater. 22, 4120 (2010).

Other groups have incorporated a secondary polymer phase to amplify the MOF signal, or even achieve increased conductivity through structural transformations to two dimensions. See S. Sachdeva et al., ACS Appl. Mater. Interface 9, 24926 (2017); and M. Campbell et al., J. Amer. Chem. Soc. 137, 13780 (2015). These strategies have proven effective to selective detection of alcohols, though changes in response have generally been limited to one order-of-magnitude. More recent work by Yassine et. al, using thin film fumarate-based MOFs, that are selective to H₂S, yielded a >100× change in capacitive response. See O. Yassine et al., Angew. Chem. Int. Ed. 55, 15879 (2016).

### SUMMARY OF THE INVENTION

The present invention is directed to an iodine sensor comprising an insulating substrate; an array of interdigitated electrodes disposed on the substrate; a coating, comprising an iodine-capture material, disposed on the array of interdigitated electrode pairs; and a frequency response analyzer for measuring the impedance response of the coating when an iodine species is absorbed in the iodine-capture material and an alternating voltage is applied to the pairs of interdigitated electrodes. The iodine-capture material can comprise a MOF, zeolite, or activated carbon material. The iodine species can comprise I₂, CH₃I, CH₂I₂, C₃H₇I, CH₂CCII, HIO₃, IO, IO₂, I₂O₂, IONO₂, ICI, HI, or HOI. The coating can have a thickness of less than 100 μm, preferably less than 10 μm, and more preferably less than 1 μm. The alternating voltage can have a frequency between 10 mHz and 1 MHz. The frequency response analyzer can further comprise a high impedance interface to enable accurate measurement of low-conductivity, low-loss coatings, especially at low frequencies.

As an example of the invention, impedance spectroscopy was used to directly detect the real-time adsorption of I₂ by a MOF-based iodine sensor. The MOF-based sensor is highly selective and responsive to I₂ gas. As an example, a sensor comprising ZIF-8 drop cast onto platinum interdigitated electrodes was exposed to gaseous I₂ at 25, 40, and 70° C. I₂ was readily detected at 25° C. in air within 720 s of exposure. The specific response is attributed to the chemical selectivity of the ZIF-8 towards I₂. Furthermore, equivalent circuit modeling of the impedance data indicates a >10⁵× decrease in ZIF-8 resistance when 116 wt % I₂ is adsorbed by ZIF-8 at 70° C. in air. Potentially interfering species, air, argon, methanol, and water were found to produce minimal changes in ZIF-8 impedance. Therefore, the selective I₂ adsorption by ZIF-8 can be leveraged to create a highly selective sensor using large changes, >10⁵×, in impedance response to enable the direct electrical detection of environmentally-relevant gaseous toxins.

### BRIEF DESCRIPTION OF THE DRAWINGS

The detailed description will refer to the following drawings, wherein like elements are referred to by like numbers.

FIG. 1 is a photograph of an uncoated interdigitated electrode IDE on a glass substrate (top), an IDE coated with ZIF-8 (middle), and an IDE coated with ZIF-8 and exposed to I₂ at 70° C. (bottom).

FIGS. 2A and 2B are graphs of the impedance response and equivalent circuit fits of IDEs uncoated, coated in ZIF-8, and coated in ZIF-8 and exposed to I₂ at 70° C. FIG. 2A is a graph of the impedance as a function of frequency. FIG. 2B is a graph of the phase angle as a function of frequency.

FIG. 3 shows the equivalent circuit used to model impedance data overlaid onto a cross-sectional schematic of the sensor, showing how the circuit elements Rₛ, R_{Z}, CPE_{Z}, R_{g}, and C_{g} spatially relate to the materials used.

FIG. 4A is a graph of the percent change (Δm/m₀) in ZIF-8 mass as a function of I₂ exposure temperature. FIG. 4B is a graph of the percent change in ZIF-8 capacitance, C_{Z}, as a function of I₂ exposure temperature. FIG. 4C is a graph of the change of the ratio of ZIF-8 resistance, R_{Z}, before I₂ exposure to that after I₂ exposure, as a function of I₂ exposure temperature. For each of these graphs, the sensors were either uncoated and exposed to I₂, coated with ZIF-8 and heated without I₂ present, or coated with ZIF-8 and exposed to I₂.

FIG. 5 is a graph of X-ray diffraction patterns of a bare IDE (plot A), IDE coated with ZIF-8 (plot B), IDE coated with ZIF-8 and exposed to I₂ at 25° C. (plot C), IDE coated with ZIF-8 and exposed to I₂ at 40° C. (plot D), and IDE coated with ZIF-8 and exposed to I₂ at 70° C. (plot E).

FIG. 6 are infrared (IR) spectra of a sensor comprising ZIF-8 drop cast onto an IDE (plot A), the sensor exposed to I₂ at 25° C. (plot B), the sensor exposed to I₂ at 40° C. (plot C), and the sensor exposed to I₂ at 70° C. (plot D).

FIG. 7A is a graph of real time measurements of the impedance magnitude for uncoated and ZIF-8-coated IDEs exposed to gaseous I₂ at 25° C. FIG. 7B is a graph of the phase angle at 100 mHz for uncoated and ZIF-8-coated IDEs exposed to gaseous I₂ at 25° C.

FIG. 8A is a graph showing the influence of I₂, air, argon, methanol, or water exposure at 40° C. on the capacitance, C_{Z}, of ZIF-8 films. FIG. 8B is a graph showing the influence of I₂, air, argon, methanol, or water exposure at 40° C. on the resistance, R_{Z}, of ZIF-8 films. No mass change was observed except for I₂, as shown in FIG. 4A.

### DETAILED DESCRIPTION OF THE INVENTION

The present invention is directed to an iodine sensor comprising a metal-organic framework (MOF), zeolite, or activated charcoal iodine-capture material disposed on interdigitated electrodes (IDEs). MOFs are hybrid organic-inorganic materials composed of a metal ion or cluster of metal ions coordinated to organic ligands, or linkers, to provide a nanoporous framework. For example, the MOF can comprise a zeolitic imidazolate framework (ZIF) material. ZIFs are composed of tetrahedrally-coordinated transition metal ions (e.g. Fe, Co, Cu, Zn) connected by imidazolate linkers and are topologically isomorphic with zeolites. ZIFs have high porosity, are resistance to thermal changes, and have robust chemical stability. Zeolites are fully inorganic, nanoporous aluminosilicate materials. For example, the zeolite can be a silver-mordenite (Ag-MOR) or other silver-exchanged zeolite. Activated carbons are fully organic forms of carbon that are processed to have small, low-volume pores with increased surface area for gas absorption. All of these materials can capture iodine and other organoiodide species, such as CH₃I, CH₂I₂, C₃H₇I, CH₂CCII, HIO₃, IO, IO₂, I₂O₂, IONO₂, ICI, HI, and HOI. These species can be present as gases or aerosolized particulates.

The invention can further comprise a frequency response analyzer for measuring the impedance response of the coating when an AC voltage is applied to the IDEs. The IDEs comprise an array of interlocking comb-shaped pairs of metallic electrodes deposited on the surface of an insulating substrate. Impedance spectroscopy measures the electrical impedance of the coated IDEs over a range of frequencies. The impedance can be related to the capacitance and conductivity of the iodine-capture material. When an alternating voltage is applied to the IDE, some energy is stored by the capacitance, and some is dissipated by the resistance effects. Therefore, the resulting current will exhibit a phase lag. The capacitance effect is known as the permittivity (or dielectric constant), and the resistive effect as dielectric loss. In materials where the dielectric loss is very small and the permittivity is large, a high impedance interface can be connected in series with the frequency response analyzer to provide a more accurate impedance measurement. The high impedance interface enables a reference measurement to be obtained on precision internal reference capacitors which are automatically substituted for the sample; a second measurement is made, this time on the sample itself. The two results are used to derive an accurate measurement of the permittivity of the sensing materal—in effect, the first measurement is used to eliminate the effects of extraneous capacitance.

Alternatively, the invention can comprise a meter for measuring the change in conductivity of the coating when an iodine species is absorbed in the iodine-capture material when a constant current, constant voltage, or swept voltage is applied to the pairs of interdigitated electrodes.

As an example of the invention, a sensor was fabricated comprising ZIF-8 coated on platinum IDEs on a glass substrate. ZIF-8 is a MOF comprised of zinc ions coordinated by four imidazolate rings, having the composition Zn(MeIM)₂. See K. S. Park et al., Proc. Nat. Acad. Sci. 103(27), 10186 (2006). Previous work has shown that the ZIF-8 is highly selective to I₂ gas through strong binding inside the framework's β-cage, where guest-framework interactions occur between the highly polarizable I₂ molecules and the 2-methylimidazole ligands. See D. F. Sava et al., J. Am. Chem. Soc. 133, 12398 (2011); and J. T. Hughes et al., J. Am. Chem. Soc. 135, 16256 (2013). Further, ZIF-8 is inexpensive and commercially available in kilogram quantities, making it an economically attractive choice for a commercial I₂ sensor. Practically, ZIF-8 is hydrophobic compared to other MOFs known to selectively absorb I₂. This hydrophobicity enables a stable background reading before I₂ is introduced. Furthermore, the pore size opening of the ZIF-8 framework closely approximates that of the I₂ molecule (head-in) and therefore can be consider optimum for the size electivity of the gas molecule.

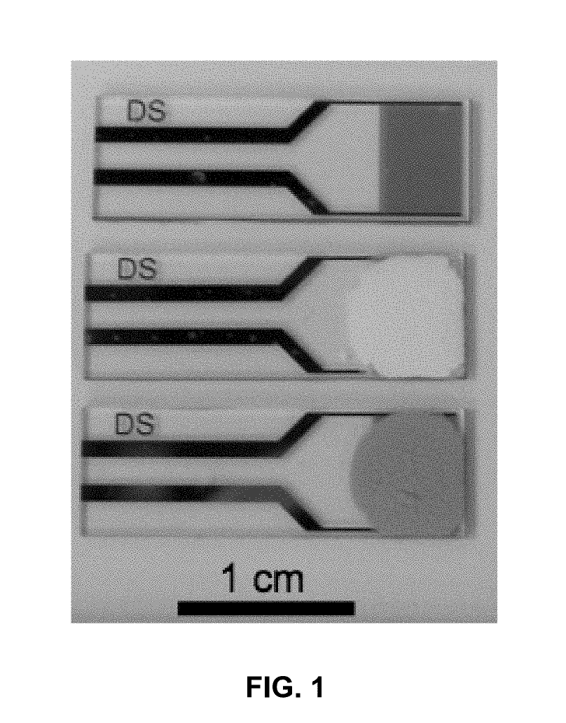

The electrodes comprised 125 pairs of platinum electrodes 250 nm thick and 10 μm wide with a 10 μm spacing between electrodes. Prior to coating with the ZIF-8 material, the IDEs were rinsed with methanol, dried under nitrogen, heated to 70° C. in air for 30 minutes, and cooled to room temperature. The IDEs were then coated with ZIF-8 using a dropcasting technique. 200 mg of ZIF-8 (Basolite Z1200, Sigma-Aldrich) was mixed with 2 mL methanol. The mixture was sealed and magnetically stirred vigorously for 30 minutes, after which 25 μL was pipetted onto the active area of the IDE. The IDE was allowed to dry at room temperature for 10 minutes, followed by heating at 70° C. on a hotplate in air for 30 minutes. This procedure consistently deposited 1.87±0.13 mg of ZIF-8 onto the IDE with a film thickness on the order of 35 μm. The sensors were exposed to I₂ at 25, 40, or 70° C. for 30 minutes, followed by heating at 70° C. in air to minimize I₂ simply absorbed to the sensor surface. I₂ vapor pressures at 25, 40, and 70° C. are 16.8, 35.2, and 124 kPa, respectively. Photographs of the sensors at each point in this process are shown in FIG. 1. The ZIF-8 covers the entire active area of the IDE, and acquires an orange-brown hue upon exposure to I₂.

Impedance spectra were recorded with a frequency spectrum analyzer connected in series with a high impedance interface, utilizing internal reference capacitors for measurements, as described above. The high input impedance of this system enabled measurement of impedances up to 10¹⁴Ω. Impedance spectra were recorded at 0 V DC and 100 mV (RMS) AC over 1 MHz-10 mHz.

### Testing of MOF-Based Sensor

The MOF-based sensor was tested under a variety of experimental conditions to examine the effects of environmental conditions on response and selectivity. These included, studying the effect of: (1) variable temperature and time of exposure to iodine gas on the sensor's response, (2) competing gas (air component) molecules on selectivity to iodine, and (3) the structural integrity of the MOF and the overall sensor after exposure to these conditions. By analyzing the resultant electrical response (impedance spectroscopy responses) under varying experimental conditions, the strength and durability of the electrical readout signal from this MOF-based sensor can be determined.

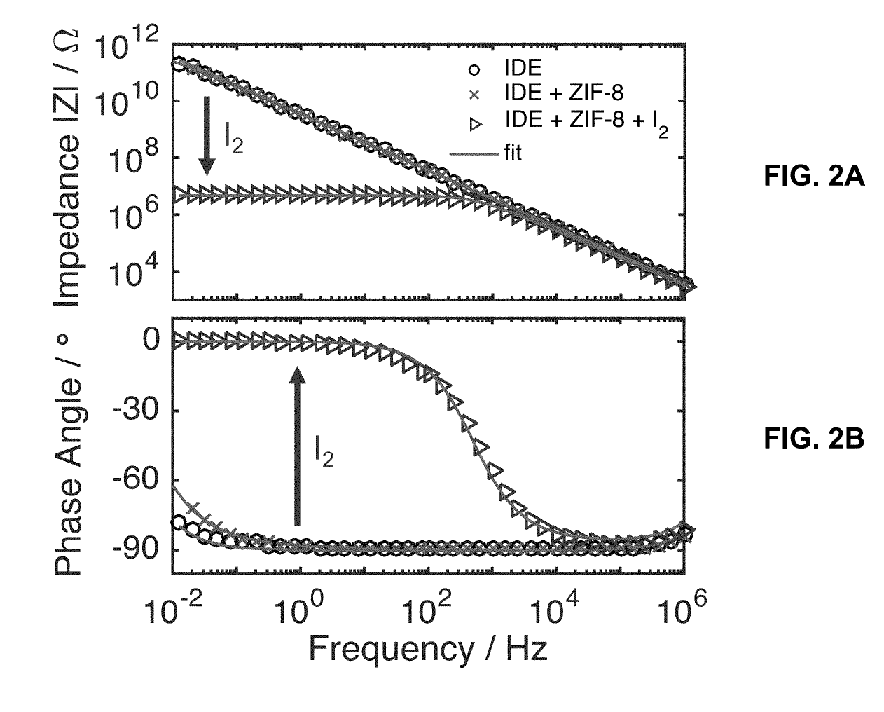

An example impedance response of these sensors is presented in FIG. 2 using the Bode format. The bare sensor has very high impedance (|Z|>10¹¹Ω at 10 mHz) and highly capacitive character (θ≈−90°), as expected for metal electrodes on a glass substrate. Upon addition of ZIF-8, the low frequency impedance only slightly decreases, and the phase angle slightly rises, consistent with reports on the high resistivity of many MOFs. See S. Achmann et al., Sensors 9, 1574 (2009); and A. Talin et al., Science 343, 66 (2014). Exposing the sensor to I₂ created a large change in both impedance and phase angle at low frequencies.

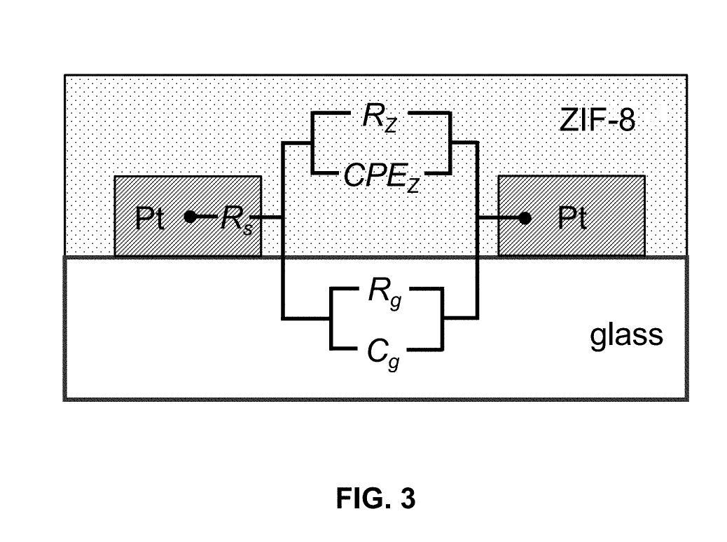

The impedance behavior of this system was modeled using an equivalent circuit to help separate the response of the glass substrate from that of the ZIF-8. This equivalent circuit, shown in FIG. 3, consists of a resistor in series with two parallel resistor-capacitor (RC) networks linked in parallel. The series resistor (R_{S}), typically 400-450Ω, relates the sum of all series resistances, predominantly the platinum electrodes on the sensor. Using this equivalent circuit, the resistance and capacitance of both the glass substrate (R_{g}, C_{g}) and the ZIF-8 (R_{Z}, CPE_{Z}) can be quantified. Here a constant phase element (CPE) is used to describe the inhomogeneity of the ZIF-8 film and the I₂ adsorbed. See R. Hurt and J. Macdonald, Solid State Ionics 20, 111 (1986); and B. Hirschorn et al., Electrochim. Acta 5, 6218 (2010). The ZIF-8 capacitance, C_{Z}, was extracted from CPE_{Z} assuming the variation in capacitance was caused by inhomogeneous distribution of I₂ through the film. See B. Hirschorn et al., Electrochim. Acta 5, 6218 (2010). For each impedance spectra, the equivalent circuit fit is also plotted in FIG. 2. To obtain the most consistent fitting results, the response of the blank sensor was recorded, equivalent circuit fitted, and the variables Rₛ, R_{g}(≈10¹²Ω) and C_{g}(≈40 pF) were fixed during subsequent analysis of a given sensor. Impedance spectra were then recorded and equivalent circuits fitted after both deposition of ZIF-8 and exposure to I₂.

### Sensor Response as a Function of Temperature

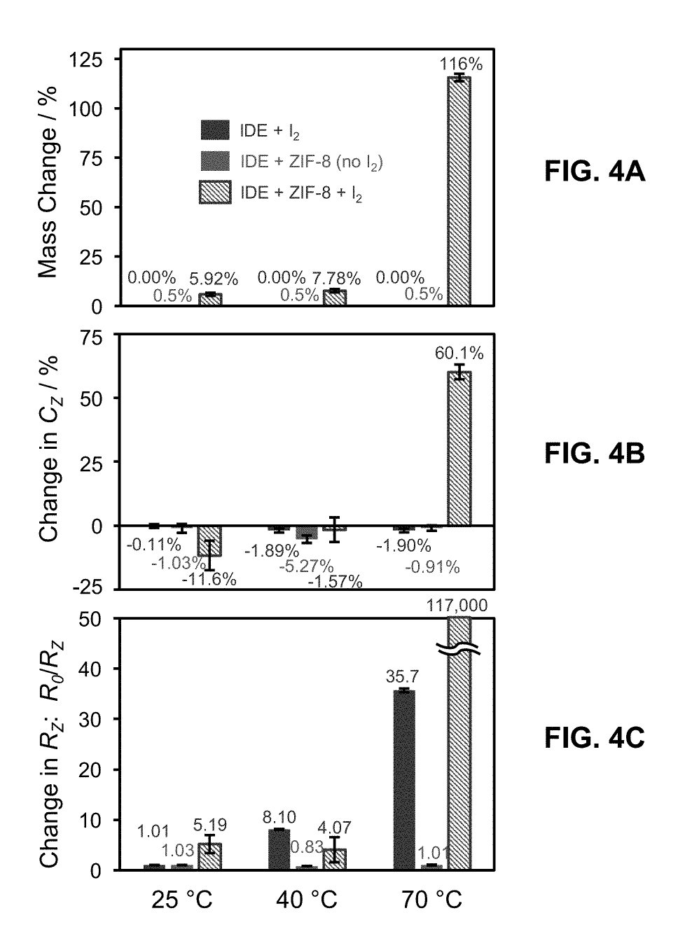

The percent change in R_{Z} and C_{Z} were determined for sensors exposed to I₂ at 25, 40, and 70° C. Additionally, two control samples were run at each temperature: (1) an uncoated sensor exposed to I₂ and (2) a ZIF-8 coated sensor thermally treated in the absence of I₂. These data are summarized in FIGS. 4A-4C.

FIG. 4A shows the percent increase in mass of each sensor after I₂ exposure. From this data, it is readily seen that both ZIF-8 and I₂ are required for the sensor to gain mass. Thermally treating the uncoated sensors had no effect on mass, and exposing an uncoated sample to I₂ resulted in no I₂ adsorption detectable via mass (±0.01 mg). The I₂ sorption of 116 wt % ZIF-8 at 70° C. is in good agreement with the maximum theoretical ZIF-8 I₂ sorption of 125 wt %, implying the ZIF-8 has nearly reached the maximum I₂ sorption capacity. See D. F. Sava et al., J. Amer. Chem. Soc. 133, 12398 (2011).

The capacitive response of the sensor, presented in FIG. 4B, shows a slight decrease in C_{Z} upon low I₂ loading levels achieved at 25 and 40° C. While the slight decrease in C_{Z} at 40° C. is statistically insignificant, the reason for the small decrease in C_{Z} observed at 25° C. is unclear. Control sensors not coated in ZIF-8, or ZIF-8 coated but not exposed to I₂ show no appreciable change in C_{Z} across all temperatures. Upon heating the sensor to 70° C., a clear divergence in responses is observed. At high I₂ loadings, C_{Z} increases 60.1% while sensors without ZIF-8 or I₂ are statistically unchanged. Thus, sorption of large amounts of I₂ by ZIF-8 significantly increased the capacitive response of the sensor. This large increase in C_{Z} is expected; the dielectric constant of vacuum (or air) is 1, while the dielectric constant of iodine varies over 3-40. See M. Simphony, J. Phys. Chem. Solids 24, 1297 (1963). To a first order approximation, exchanging all empty space in the ZIF-8 structure for iodine would therefore be expected to significantly increase the capacitance (C_{Z}).

The most profound changes in response are seen in terms of the ZIF-8 resistance, R_{Z}. Changes in R_{Z} are plotted in FIG. 4C as a direct ratio of the ZIF-8 resistance before (R₀) to that after (R_{Z}) I₂ exposure (1.00=unchanged). Uncoated sensors were found to maintain a constant R_{Z} at 25° C., though at higher temperatures, enough I₂ coated the sensor surface such that the device resistance decreased by a factor of 35.7. Sensors not exposed to I₂ showed essentially no change in R_{Z}, regardless of exposure temperature. Sensors coated with ZIF-8 and exposed to I₂, however, revealed a resistance which decreased with increased I₂ loadings. In fact, at the highest I₂ loading, achieved at 70° C., greater than 10⁵× decrease in R_{Z} was observed.

The variability of R_{Z} at low temperatures is significant; at 25 and 40° C., uncertainty was 34% and 60%, respectively, of the reported value. This uncertainty is dominated by experimental reproducibility; contributions from impedance accuracy (0.2%) and fitting uncertainty for R_{Z} (3-6%) are minimal. It is likely that the exact distribution of I₂ in the sensor, in terms of both molecular location in the ZIF-8 (sodalite cages vs. absorbed to surface) and penetration depth relative to the Pt electrodes, greatly influences the recorded values of R_{Z}. At 70° C., uncertainty is still 60%, though this uncertainty is insignificant compared to the 5 orders of magnitude change in response. However, the evaluation of R_{Z} unequivocally demonstrates that sorption of I₂ into ZIF-8 profoundly changes the impedance response at high I₂ loadings.

### MOF Structural Analysis and Sensor Response after Exposure to Iodine

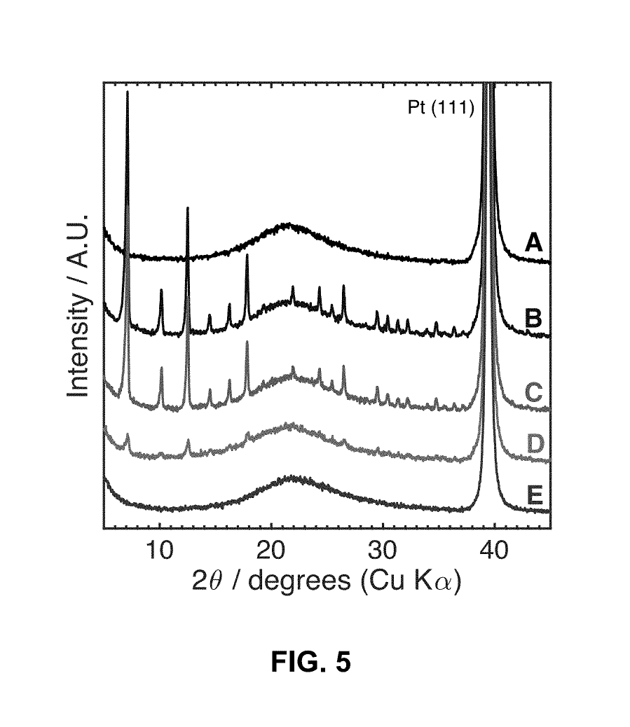

To ascertain changes in ZIF-8 crystal structure upon I₂ sorption, powder X-ray diffraction (XRD) was performed on uncoated, ZIF-8 coated, and ZIF-8 coated and I₂ exposed samples. The resulting data is plotted in FIG. 5. The XRD data for uncoated IDEs, shown as plot A, display a broad peak near 22° attributed to the glass substrate and a sharp Pt (111) peak at 40°. Upon drop casting and drying ZIF-8 at 70° C., the powder pattern characteristic of ZIF-8 is observed in plot B. Exposure to I₂ at 25° C. resulted in no appreciable change in XRD, while exposure at 40° C. showed a decrease in ZIF-8 peak intensity, and 70° C. exposure displayed a complete absence of ZIF-8 peaks, as shown in plots C, D, and E, respectively. Aside from glass, Pt, and ZIF-8, no additional peaks were observed. From this data, it is concluded that the long-range order of the ZIF-8 has been lost at the high I₂ loadings achieved during the 70° C. exposure. Based on previous work, it is anticipated that short-range order is maintained under these conditions. See K. Chapman et al., J. Amer. Chem. Soc. 133, 18583 (2011).

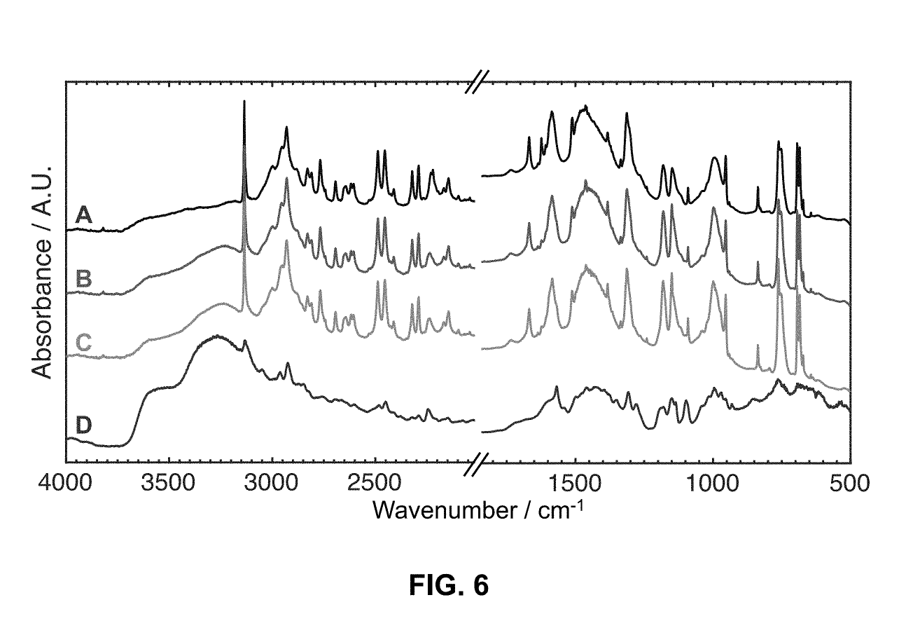

As shown in FIG. 6, the samples were also interrogated by IR spectroscopy to understand the chemical bonding environment. Similar to the XRD measurements, the ZIF-8 coated IDEs display IR spectra as expected for ZIF-8, as shown in plot A. See U. Tran et al., ACS Catal. 1, 120 (2011). As sensors are exposed to I₂ at 25° C. (plot B) or 40° C. in (plot C), a shoulder near 3600 cm⁻¹ and a broad peak at 3254 cm⁻¹ develop. These are related to increased number of terminal N—H groups upon breaking the long-range order of the ZIF-8 structure, and N—H interactions with adsorbed iodine species. Exposure to I₂ at 70° C. resulted in both broadening and decreased intensity in most peaks, with the exception of those at 3600 and 3254 cm⁻¹, whose relative intensities increased, as shown in plot D. The general broadening and suppression of peak intensity is consistent with IR literature reports on the amorphization of the ZIF-8 crystal structure. See Y. Hu et al., Chem. Commun. 47, 12694 (2011); and Y. Hu et al., J. Amer. Chem. Soc. 135, 9287 (2013).

It has been previously shown that amorphization of the ZIF-8 structure enhances I₂ capture and retention without destruction of the local structure surrounding the captured I₂. See K. Chapman et al., J. Amer. Chem. Soc. 133, 18583 (2011); and T. Bennett et al., Chem. Eur. J. 19, 7049 (2013). Attempts to remove I₂ from the 70° C. samples using moderate vacuum (<1 mTorr) under heat (70° C.) were only successful in removing 28 wt % of ZIF-8 in I₂, leaving 88 wt %. This is in good agreement with the 125 wt % maximum capacity of I₂ in ZIF-8, with ≈100 wt % efficiently contained within the sodalite cages and ≈25 wt % simply adsorbed to the surface. See D. F. Sava et al., J. Amer. Chem. Soc. 133, 12398 (2011). Impedance spectra and XRD patterns of these evacuated samples were nominally unchanged from before, indicating irreversible structural and electrical responses. Therefore, the sensor can be an integrating sensor whereby the present response is a function of the total dose of the gas absorbed, not the present concentration of the gas in the environment.

It is hypothesized that the sorption of iodine enables new, faster charge transfer pathways, resulting in significantly lower impedances and R_{Z} values. Some reports have shown complex, interconnected networks of polyiodides formed in porous organic cages upon sorption of I₂. See T. Hassel et al., J. Amer. Chem. Soc. 133, 14920 (2011). Such a network of I₂/I⁻/I₃⁻ would be expected to significantly decrease the sensor resistance through facile charge transfer pathways. See T. Hassel et al., J. Amer. Chem. Soc. 133, 14920 (2011); and Y.-Q. Hu et al., Chem. Eur. J. 23, 8409 (2017). That such large decreases in resistance are not seen until high I₂ loadings also supports this idea, and is consistent with previous work where it was observed that I₂ was strongly bound in type I sites filled preferentially at low I₂ loadings, followed by less tightly bound I₂ farther out in the pore. See D. F. Sava et al., J. Amer. Chem. Soc. 133, 12398 (2011).

### Sensor Response as a Function of Time

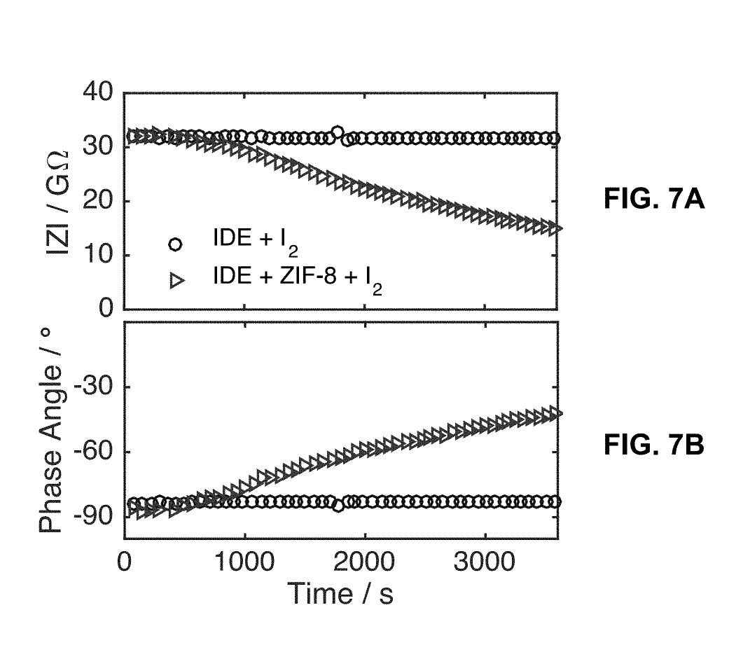

These sensors were successfully used to detect I₂ in real time at room temperature in air. A frequency of 100 mHz was chosen to continuously monitor the impedance and phase angle, with the resulting data plotted in FIG. 7. Here it is readily seen that the impedance magnitude, |Z|, decreases with I₂ sorption, whereas the phase angle increases, consistent with previous results. Within 1200 s, I₂ is detected with a S/N of 3 using |Z| and assuming 2% error. Analyzing the real part of the impedance (Z′=|Z|·cos θ), I₂ detection can be achieved in 720 s (S/N=3).

Initially, larger changes in impedance can be seen at lower frequencies. Unfortunately, measurement times at lower frequencies start to eclipse the response of the device; measurements at 10 mHz require near 600 s, while those at 100 mHz require no more than 60 s. Optimization of sample geometry, thinning ZIF-8 film thickness from 35 μm to <1 μm, should increase the sensitivity of the sensor by requiring a lower absolute mass of captured I₂ to create the same impedance response.

It is worth noting that unlike traditional sensors, which often display a reversible electrical response to a chemical stimulus, the present impedance value of these sensors relates the present I₂ loading in the sensor, and not the environmental level. Thus, this sensor is an integrating sensor that detects whether the sensor has ever been exposed to I₂ in its lifetime. Because of the preferential sorption of I₂ in ZIF-8, it should be possible to detect the presence of I₂ at extremely low concentrations, given a long enough exposure time.

### Sensor's Iodine Selectivity Versus Competing Gas Species

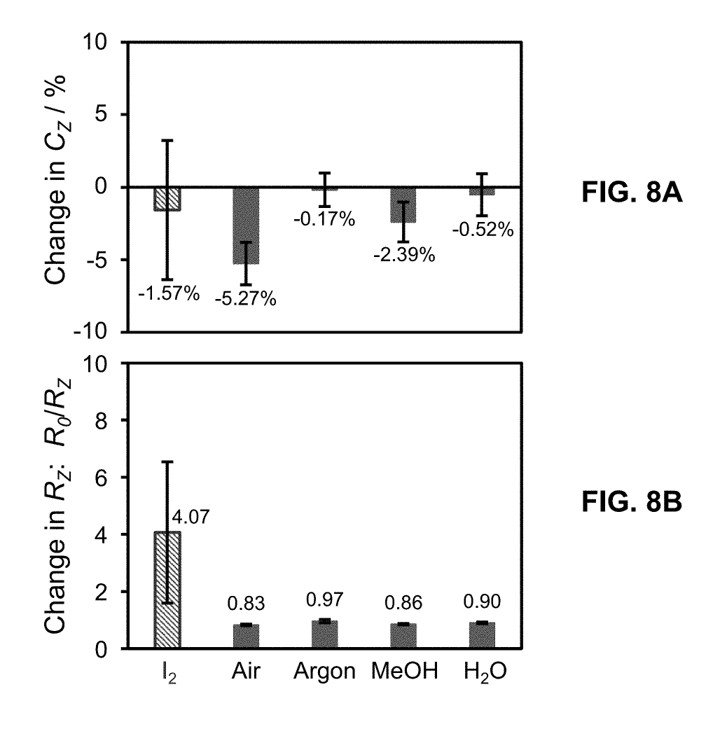

One of the most attractive aspects of using MOFs for chemical sensors is the chemical tunability of the structures and how these influence the selective sorption of various species, minimizing interfering responses. In FIG. 4 it was demonstrated that ZIF-8 shows minimal impedance response in air at all temperatures tested. To further probe the chemical selectivity of the sensor, argon, methanol, and water were tested at 40° C. The resulting changes in capacitance, C_{Z}, and resistance, R_{Z} are plotted in FIGS. 8A and 8B, respectively, and compared to those of I₂ and air. Unsurprisingly, no appreciable change in C_{Z} was observed at 40° C. across all chemicals tested, including I₂. The resistive response, R_{Z}, also showed small changes for the possible interfering species air, argon, methanol, and water. These increases in R_{Z} are quite small in comparison to the 4× decrease in R_{Z} observed upon I₂ exposure. Further, MOFs can be specifically tuned with selectivity to a wide variety of iodine chemical species. Additionally, the MOF film thickness can be decreased and the sensor capacitance maximized to enable faster detection of trace I₂.

The present invention has been described as a sensor for the direct detection of iodine. It will be understood that the above description is merely illustrative of the applications of the principles of the present invention, the scope of which is to be determined by the claims viewed in light of the specification. Other variants and modifications of the invention will be apparent to those of skill in the art.

We claim:

1. An iodine sensor, comprising:
an insulating substrate;
an array of interdigitated electrodes disposed on the substrate;
a coating, comprising an iodine-capture material, disposed on the array of interdigitated electrode pairs; and
a frequency response analyzer for measuring the impedance response of the coating when an iodine species is absorbed in the iodine-capture material and an alternating voltage is applied to the pairs of interdigitated electrodes.

2. The iodine sensor of claim 1, wherein the iodine-capture material comprises a MOF material.

3. The iodine sensor of claim 2, wherein the MOF material comprises a zeolitic imidazolate framework material.

4. The iodine sensor of claim 3, wherein the zeolitic imidazolate framework material comprises ZIF-8.

5. The iodine sensor of claim 1, wherein the iodine-capture material comprises a zeolite or activated carbon.

6. The iodine sensor of claim 5, wherein the zeolite comprises silver-mordenite.

7. The iodine sensor of claim 1, wherein the iodine species comprises an iodine-containing gas or aerosol.

8. The iodine sensor of claim 1, wherein the iodine species comprises I₂, CH₃I, CH₂I₂, C₃H₇I, CH₂CCII, HIO₃, IO, IO₂, I₂O₂, IONO₂, ICI, HI, or HOI.

9. The iodine sensor of claim 1, wherein the coating has a thickness of less than 100 μm.

10. The iodine sensor of claim 9, wherein the coating has a thickness of less than 10 μm.

11. The iodine sensor of claim 10, wherein the coating has a thickness of less than 1 μm.

12. The iodine sensor of claim 1, wherein the alternating voltage has a frequency between 10 mHz and 1 MHz.

13. The iodine sensor of claim 1, further comprising a high impedance interface connected in series with the frequency response analyzer.

14. The iodine sensor of claim 1, wherein the sensor has an operating temperature of less than 70° C.

15. The iodine sensor of claim 1, wherein the iodine sensor is an integrating sensor.

16. An iodine sensor, comprising:
an insulating substrate;
an array of interdigitated electrodes disposed on the substrate;
a coating, comprising an iodine-capture material, disposed on the array of interdigitated electrode pairs; and
a meter for measuring a change in conductivity of the coating when an iodine species is absorbed in the iodine-capture material and a constant current, constant voltage, or swept voltage is applied to the pairs of interdigitated electrodes.
